(19) United States
(12) Patent Application Publication
(10) Pub. No.: US 2019/0343452 A1
(43) Pub. Date: Nov. 14, 2019

(54) BRUXISM MONITORING AND PREVENTION SYSTEM

(71) Applicant: EMRO Technologies, LLC, Austin, TX (US)

(72) Inventor: Brian Ellspermann, Austin, TX (US)

(21) Appl. No.: 16/406,981

(22) Filed: May 8, 2019

Related U.S. Application Data
(60) Provisional application No. 62/668,923, filed on May 9, 2018.

(51) Int. Cl. A61B 5/00

(52) U.S. Cl. CPC A61B 5/4557; A61B 5/0002; A61B 5/6817; A61B 5/7405

(57) ABSTRACT

A bruxism monitoring and prevention system includes a bruxism detection subsystem that is dimensioned to be positioned at least partly in an ear canal of a patient. The bruxism detection subsystem detects, when the bruxism detection subsystem is positioned at least partly in the ear canal, a change in a size of the ear canal of the patient and, in response, generates a bruxism event detection signal. A bruxism notification subsystem is coupled to the bruxism detection subsystem. The bruxism notification subsystem receives the bruxism event detection signal generated by the bruxism detection subsystem and, in response, generates a tone that is configured to be audible to the patient.

### CROSS-REFERENCE TO RELATED APPLICATIONS

This application claims priority to U.S. Provisional Application Ser. No. 62/668,923, filed May 9, 2018, entitled “Bruxism Monitoring And Prevention System,” Attorney Docket No. 70694.3PV01, the disclosure of which is incorporated herein by reference in its entirety.

### BACKGROUND

The present disclosure relates generally to bruxism, and more particularly to monitoring and detecting bruxism.

Bruxism is a condition in which a patient grinds, gnashes, or clenches their teeth, and may be performed unconsciously when the patient is awake (awake bruxism) and/or during sleep (sleep bruxism). Sleep bruxism is considered a sleep-related movement disorder, and patients who clench or grind their teeth during sleep are more likely to have other sleep disorders, including snoring and sleep apnea. While mild bruxism may not require treatment, in many patients bruxism can be frequent and severe enough to lead to jaw disorders, headaches, damaged teeth and other problems. Conventional treatments for bruxism include mouth guards or mouth splints that the patient wears to even out the pressure across their jaw and create a physical barrier between their upper and lower teeth to protect them from further damage, treatments for stress/anxiety (e.g., yoga, deep breathing, massage, etc.) that may be the root cause of the bruxism, reconstructive dental treatments (e.g., false teeth, overlays, crowns) to correct dental issues (e.g., misaligned, cracked, and crooked or missing teeth), and medication such as muscle relaxants. Each of these treatments suffers from several deficiencies and, as a result, the conventional treatments for bruxism produce less than desirable results.

Accordingly, it would be desirable to provide an improved bruxism treatment system.

### SUMMARY

According to one embodiment, a bruxism monitoring and prevention system includes a bruxism detection subsystem that is dimensioned to be positioned at least partly in an ear canal of a patient, wherein the bruxism detection subsystem is configured: detect, when the bruxism detection subsystem is positioned at least partly in the ear canal, a change in a size of the ear canal of the patient; and generate, in response to detecting the change in the size of the ear canal, a bruxism event detection signal; and a bruxism notification subsystem that is coupled to the bruxism detection subsystem, wherein the bruxism notification subsystem is configured to: receive the bruxism event detection signal generated by the bruxism detection subsystem; and generate, in response to receiving the bruxism event detection signal, a tone that is configured to be audible to the patient

### BRIEF DESCRIPTION OF THE DRAWINGS

FIG. 1 is a schematic view illustrating an embodiment of an Information Handling System (IHS).

FIG. 2 is a perspective view illustrating an embodiment of a bruxism monitoring and prevention system provided in a mouth guard.

FIG. 3 is a perspective view illustrating an embodiment of a bruxism monitoring and prevention system that utilized a mouth guard that may be similar to the mouth guard illustrated in FIG. 2, along with an optical feedback eye mask.

FIG. 4 is a perspective view illustrating an embodiment of a bruxism monitoring and prevention system provided in an optical feedback eye mask.

FIG. 5 is a perspective view illustrating an embodiment of a bruxism monitoring and prevention system provided in an ear bud.

FIG. 6 is a perspective view illustrating another embodiment of a bruxism monitoring and prevention system provided in an ear bud.

FIG. 7 is a front/back/side/top view illustrating another embodiment of a bruxism monitoring and prevention system provided in an ear bud.

FIG. 8 is a front/back/side/top view illustrating an embodiment of the bruxism monitoring and prevention system provided in the ear bud illustrated in FIG. 7.

FIG. 9 is a front/back/side/top view illustrating an embodiment of a case for the bruxism monitoring and prevention system provided in the ear bud illustrated in FIGS. 7 and 8.

FIG. 10 is a perspective view illustrating an embodiment of the case for the bruxism monitoring and prevention system illustrated in FIG. 9.

FIG. 11 is a front/back/side/top view illustrating an embodiment of the case for the bruxism monitoring and prevention system illustrated in FIG. 9.

FIG. 12 is a perspective view illustrating an embodiment of a patient using the bruxism monitoring and prevention system provided in the ear bud illustrated in FIGS. 7 and 8.

FIG. 13 is a perspective view illustrating an embodiment of a patient using the bruxism monitoring and prevention system provided in the ear bud illustrated in FIGS. 7 and 8.

FIG. 14 is a perspective view illustrating an embodiment of a patient using the bruxism monitoring and prevention system provided in the ear bud illustrated in FIGS. 7 and 8, and prior to a bruxism event.

FIG. 15 is a perspective view illustrating an embodiment of a patient using the bruxism monitoring and prevention system provided in the ear bud illustrated in FIGS. 7 and 8, during to a bruxism event.

FIG. 16 is a perspective view illustrating an experimental embodiment of the bruxism monitoring and prevention system of the present disclosure.

FIG. 17 is a schematic view illustrating an experimental embodiment of the bruxism monitoring and prevention system of the present disclosure.

FIG. 18 is a schematic view illustrating an experimental embodiment of the bruxism monitoring and prevention system of the present disclosure.

FIG. 19 is a schematic view illustrating an experimental embodiment of the bruxism monitoring and prevention system of the present disclosure.

FIG. 20 is a schematic view illustrating an experimental embodiment of the bruxism monitoring and prevention system of the present disclosure.

FIG. 21 is a schematic view illustrating an experimental embodiment of the bruxism monitoring and prevention system of the present disclosure.

FIG. 22 is a schematic view illustrating an experimental embodiment of the bruxism monitoring and prevention system of the present disclosure.

### DETAILED DESCRIPTION

For purposes of this disclosure, a computing device may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a computing device may be a personal computer (e.g., desktop or laptop), tablet computer, mobile device (e.g., personal digital assistant (PDA) or smart phone), server (e.g., blade server or rack server), a network storage device, or any other suitable device and may vary in size, shape, performance, functionality, and price. The computing device may include random access memory (RAM), one or more processing resources such as a central processing unit (CPU) or hardware or software control logic, ROM, and/or other types of nonvolatile memory. Additional components of the computing device may include one or more disk drives, one or more network ports for communicating with external devices as well as various input and output (I/O) devices, such as a keyboard, a mouse, touchscreen and/or a video display. The computing device may also include one or more buses operable to transmit communications between the various hardware components.

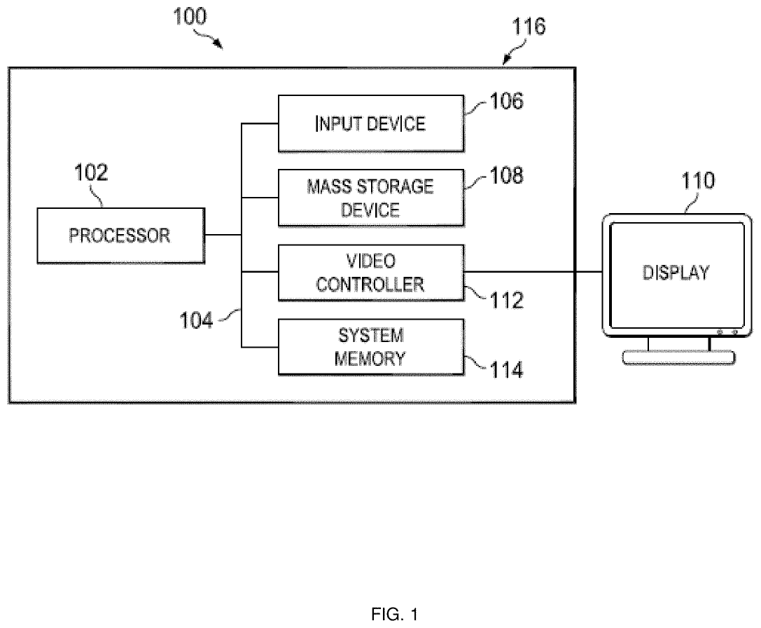

In one embodiment, computing device 100, FIG. 1, includes a processor 102, which is connected to a bus 104. Bus 104 serves as a connection between processor 102 and other components of computing device 100. An input device 106 is coupled to processor 102 to provide input to processor 102. Examples of input devices may include keyboards, touchscreens, pointing devices such as mouses, trackballs, and trackpads, and/or a variety of other input devices known in the art. Programs and data are stored on a mass storage device 108, which is coupled to processor 102. Examples of mass storage devices may include hard discs, optical disks, magneto-optical discs, solid-state storage devices, and/or a variety other mass storage devices known in the art. Computing device 100 further includes a display 110, which is coupled to processor 102 by a video controller 112. A system memory 114 is coupled to processor 102 to provide the processor with fast storage to facilitate execution of computer programs by processor 102. Examples of system memory may include random access memory (RAM) devices such as dynamic RAM (DRAM), synchronous DRAM (SDRAM), solid state memory devices, and/or a variety of other memory devices known in the art. In an embodiment, a chassis 116 houses some or all of the components of IHS 100. It should be understood that other buses and intermediate circuits can be deployed between the components described above and processor 102 to facilitate interconnection between the components and the processor 102. As will be appreciated by one of skill in the art in possession of the present disclosure, the computing device may be utilized with and/or integrated with the bruxism monitoring and prevention system discussed below to enable any of the functionality described herein.

The inventor of the present disclosure has recognized how the diameter of the ear canal decreases in response to how hard the jaw is clenched, and that this effect may be utilized to detect bruxism, as patients with bruxism tend to clench their jaws very hard, often hard enough to cause damage to their teeth. Embodiments of the bruxism monitoring/prevention device for treating bruxism based on this concept will calibrate itself to the size of patient's ear canal, and then operate to sense contractions resulting from the patient clenching their jaw, and relay information about the clench of the patient's jaw to a connected device for logging the occurrence of the jaw clenching, and produce a tone that is audible to the patient and that is configured to remind the patient to unclench their jaw and relax. The bruxism monitoring/prevention device may work while the user is awake or asleep, and data collected based on the logging operations discussed above will be available for analysis by patients or medical professionals in a user-friendly format, which allows for diagnosis, effective training to reduce jaw clenching, and confirmation that the training is working. Several methods may be utilized detecting a bruxism event, including: using a microphone to detect/measure a particular sound that grinding teeth produce, detecting the force with which a jaw muscle contracts using an electromyographic sensor, measuring the physical decrease in ear canal diameter caused by the contraction using piezoelectric or resistive sensors, and/or other methods discussed below.

Microphone-Based Detection of Bruxism Events

In some embodiments of the present disclosure, a microphone may be utilized to detect and/or record the sound of teeth grinding in order to detect bruxism. Devices incorporating this concept may include a microcontroller powerful enough to compare the incoming soundwave measured from inside the ear canal with a stored waveform in order to determine whether the sound detected by the microphone was caused by bruxism. This data may then be sent to a receiving device (e.g., the patient's mobile phone, which may be paired with the microphone/microcontroller) for logging, and an audible tone may be produced in response to detecting the grinding of teeth in an attempt to prompt the patient to stop grinding their teeth. As will be appreciated by one of skill in the art in possession of the present disclosure, the tone discussed above may be removed from any recording by the microphone as this would interfere with the detection algorithm, or the microphone may be disabled while a corresponding speaker produces the tone.

Electromyograph

In some embodiments of the present disclosure, an electromyographic may be utilized to allow for the detection of jaw muscle tension in the ear canal, measuring electrical activity below the skin using conductive pads and a integrated circuit. However, current electromyographic systems use relatively large hardware and require the skin in contact with the sensors to be clean, and thus the patient may be required to swab their ear canals with alcohol (or a similar solvent) in order to remove cerumen and skin cells and allow for accurate muscle tension detection.

Pressure Sensor

In a plurality of different embodiments, a variety of physical pressure sensing devices may be utilized to detect bruxism according to the teachings of the present disclosure. For example, a force-sensitive resistor may be provided that is configured to detect clenching force as pressure against the sensor. In another example, an air- or fluid-filled bulb may be compressed against a barometric pressure sensor, allowing for the detecting of a clenching force as a pressure increase against the sensor.

Force Sensitive Resistor

In some embodiments, a force sensitive resistor that experiences changes in resistance relative to an applied force may be utilized to detect bruxism according to the teachings of the present disclosure. For example, such a force sensitive resistor may be provided via a conductive polymer grid located on a flexible substrate. While one of skill in the art in possession of the present disclosure will recognize that such force sensitive sensors are not particularly precise, the inventor of the present disclosure has found that force sensitive sensors may be calibrated to a patient to allow for the detection of bruxism events. For example, the bruxism monitoring/prevention device would be provided with a primarily rigid chassis, with a semi-flexible portion that allow for deformation, which may be provided by jaw muscle clenching in order to allow a bruxism event to be detected by the force sensitive resistor.

MEMS Pressure Sensor

In some embodiments, a micro-electro-mechanical (MEM) system may be utilized to detect bruxism according to the teachings of the present disclosure, and one of skill in the art in possession of the present disclosure will recognize that such MEMS systems provide relatively small form factors that enable at least some of the embodiments discussed below. Many MEMs systems take advantage of the piezoelectric effect (e.g., the effect that pressure has on certain kinds of crystal and that causes those crystals to produce electricity when pressure is applied to them, as well as change shape when power is applied) in order to allow very precise detection of pressure changes in order to detect bruxism events. For example, a MEMS pressure sensor may detect changes in current produced when a thin piece of piezoelectric material is deformed by applied pressure, and may be utilized by at least some of the devices of the present disclosure via a small hollow bulb that may be inserted into the ear of a patient. A pressure sensor may be attached to the bulb in such a way that when the bulb is compressed by the decrease in ear canal diameter caused by jaw clenching, air is forced from the bulb into the sensor. That sensor may then be read by a microcontroller, and the amount of pressure determined and relayed to a remote system (e.g., a dedicated computing device, a patient mobile phone, etc.) for logging, while a speaker may play a noise into the user's ear canal in an attempt to remind the patient to relax their jaw.

As will be appreciated by one of skill in the art in possession of the present disclosure, MEMS systems are relatively small devices that take advantage of the electrical properties of relatively small mechanical parts. For example, the use of piezoelectric materials on silicon substrates (e.g., the same silicon substrates used to manufacture computing device subsystems) enables the mass production of miniaturized devices, with only a relatively small amount of material required to generate a usable signal. In a specific example, MEMS piezoresistive pressure sensors may be configured to accurately measure barometric pressure by detecting changes in the electrical resistance of a very thin piece of silicon, with that electrical resistance change amplified and output in a usable form. As such, a relatively thin piece of material may span an etched cavity that is hollow (or sometimes evacuated and sealed), and when pressure is applied to the front surface of the thin material it deforms slightly into the hollow cavity behind it. This deformation stretches the material, which changes its electrical properties, and the monitoring of those electrical properties allows their change to be detected immediately in response to relatively small amounts of pressure. This change may then be converted to a usable signal, suitable for recording by a microcontroller and further processing in the bruxism monitoring/prevention devices discussed herein.

Speaker

As discussed above, the bruxism monitoring/prevention device may be provided with a speaker that is configured to produce audible tones that attempt to remind the patient to stop clenching their jaw/grinding their teeth. In light of the size of some embodiments of the bruxism monitoring/prevention system of the present disclosure, a balanced armature receiver may be utilized that provides a small form factor while being readily available for consumer applications.

Microcontroller

As discussed above, a microcontroller (e.g., provided by the processor 102 discussed above with reference to FIG. 1) may be utilized to process and relay signals to a receiving device (e.g., the patient mobile phone and/or other computing devices discussed above). In different examples, the microcontroller may be provided with a form factor that is small enough to fit inside the ear canal, and may be designed to use as little power as possible. For example, the microcontroller may be configured to operate at a relatively low-power mode that provides power savings associated with a battery life of a battery in the bruxism monitoring/prevention device (e.g., when the patient is not grinding their teeth), while only “waking” to perform its necessary functions when bruxism events are detected.

Bluetooth

In some embodiments, the latest BLUETOOTH® 5.0 standard may be implemented in the bruxism monitoring/prevention device, and one of skill in the art in possession of the present disclosure will recognize that this wireless communication standard uses very low amounts of power, while having a relatively long wireless communication range. Wireless hardware for such wireless communication system(s) may be tightly integrated with the microcontroller discussed above (e.g., on the same circuit board.)

Battery

As discussed above, a rechargeable battery (e.g., a lithium-ion battery) may be provided in the bruxism monitoring/prevention device, and in some examples may include a capacity of around 50-100 mAh, which allows for the powering of the miniaturized electronics utilized in the bruxism monitoring/prevention device for at least a full night of use. Furthermore, bruxism monitoring/prevention device storage case may be configured to recharge the bruxism monitoring/prevention device, and may include its own dedicated battery that is configured to charge the bruxism monitoring/prevention device several times before the storage case requires recharging, which allows for use of the bruxism monitoring/prevention device on the go and for a plurality of days.

The bruxism monitoring/prevention devices of the present disclosure maybe manufactured in a relatively inexpensive and reliable device that can be worn almost imperceptibly while sleeping. In many embodiments, minimal preparation of the ear canals of the patient is required, which is enabled via the detection of ear canal diameter changes using pressure sensors and a microcontroller/battery to detect muscle contractions, and in some cases transmit monitored data generated from such operations to a connected device such as a patient's mobile phone.

Force Sensitive Resistor

In some embodiments of the present disclosure that utilize the force sensitive resistor discussed above, the bruxism monitoring/prevention device may detect pressure increases on a specific part of a cylindrical surface, with data generated from those detected pressure difference transmitted by a microcontroller via BLUETOOTH wireless communications to a receiving device (e.g., a computing device, patient mobile phone, etc.), which may then operate to determine whether or not to issue an audible tone to the patient in an attempt to stop the patient from grinding their teeth. As will be appreciated by one of skill in the art in possession of the present disclosure, this type of pressure measuring technique may allow the bruxism monitoring/prevention device to be made “acoustically invisible”, that is, the patient may be able to hear the world around them while using the bruxism monitoring/prevention device.

MEMS Pressure Sensor

Some embodiments of the present disclosure may utilize the measurement of changes in ear canal diameter via detecting increases in pressure in a sealed chamber. For example, a sealed bulb connected to a MEMS pressure sensor may be configured to push air into the sensor in proportion to the strength of a jaw muscle contraction by a patient, and a microcontroller may transmit data generated from the sensor to a receiving system (e.g., the patient mobile phone and/or other computing device) that may be configured to generate a sound that attempts to alert the patient to their subconscious jaw clenching. As will be appreciated by one of skill in the art in possession of the present disclosure, such techniques may provide a relatively accurate method for measuring jaw clenching. However, in some embodiment, the ear buds discussed below for providing embodiments of the bruxism monitoring/prevention system of the present disclosure may be configured to muffle incoming sound, which one of skill in the art in possession of the present disclosure will recognize may provide benefits for patients sleeping or otherwise using the bruxism monitoring/prevention device at night time.

Microcontroller Board

A circuit board including a microcontroller, Bluetooth wireless communications circuitry, and/or other subsystems may be provided that is configured to fit within or adjacent to the ear canal of a patient, and the components/subsystems may be selected to be as low-power as possible. Furthermore, the microcontroller may be configured to control a speaker, receive analogue data from a pressure sensor, and wirelessly transmit data to a receiving device. The circuit board may also be configured to manage charge and discharge of a connected rechargeable battery.

Power

The bruxism monitoring/prevention device may be sized to include a rechargeable power supply (e.g., a lithium-ion battery). For example, the capability of the battery may be around 50-100 mAh, but may vary based on the sizes/power requirements of the other components utilized in the bruxism monitoring/prevention device, and may be configured to be able to provide power for at least a full night of use of the bruxism monitoring/prevention device. Recharging of that battery may be managed by the microcontroller/circuit board, and as discussed herein may be provided via an included storage case that may provide surface contacts that are configured to connect to the earbuds and a USB port in order to allow its connection to a wall adapter. In some embodiments, the case may include its own battery that is configured to recharge the earbuds fully several times while access to a power source is limited/unavailable.

Use Scenario

A patient diagnosed with bruxism may insert earbuds that provide an embodiment of the bruxism monitoring/prevention device into their ears. After approximately five seconds, the sensors in the earbuds may automatically calibrate to the resting (i.e., with jaw unclenched) ear canal diameter of the patient, and a calibrated sound may be provided to indicate that this step is complete of the treatment is complete.

When the patient bites down relatively hard, they will be able to trigger a tone that rises in volume the harder and longer they bite, and the patient may practicing biting a few times to help align the subconscious mind with the audible feedback that attempts to prevent jaw clenching. The patient may then subsequently insert the earbuds in their ears when they go to sleep, and may be awakened by the familiar tone when they clench their jaw, grind their teeth during sleep. Recognizing the tone, the patient may relax and fall back asleep. Following sleep, the patient may be awakened by their phone playing an alarm through the earbuds, and the patient may be presented with a bruxism log for the night that details bruxism events detected via the bruxism monitoring/prevention device (e.g., seven bruxism events may be identified, detailing varying force and duration of the bruxism events, all but one of which the patient slept through.) In some embodiments, it may be noted that, as the earbuds produced their tone, the patient relaxed their jaw muscles, which may result in the patient feeling less tension in their neck and jaw as compared to previous nights. Use of the bruxism monitoring/prevention device over weeks may reduce the pain caused by the user's clenching (e.g., in proportion to the noted reduction in the duration of their bruxism events according to the logs discussed above.)

As such, some embodiments of the present disclosure describe a bruxism monitoring/prevention device that includes a pair of wireless “ear-bud” that are configured to fit within a user's ear (i.e., the outer portion of the ear canal), and that operate to detect physical pressure changes that result from muscle contractions around the ear canal that are due to clenching/grinding of the jaw, as well as emit an audible tone when such pressure changes are detected. Such pressure changes may be detected using a force sensitive resistor, a MEMS pressure sensor, compression sensing devices, etc., that may be provided on the ear bud (e.g., on an outer surface of the ear bud that engages the ear canal, in a flexible substrate on the ear bud (e.g., a foam or silicon ear bud cover), etc.) The sensing of pressure changes/compression of the muscle around the ear canal due to jaw clenching/grinding differs from prior art electrodes that are used to detect electrical signals generated from muscle contraction.

The bruxism monitoring/prevention device may wirelessly communicate (e.g., via Bluetooth) with a computing device (e.g., a mobile phone), and includes software that provides for the sending of data associated with the detected pressure changes and/or emitted audio tones to the computing device, which allow software on the computing device to record statistics related to jaw clenching/grinding. Furthermore, software on the computing device may allow the user control operation of the device (e.g., selecting a particular audio tone, setting a level of the pressure change that will result in an audio tone, calibrate the system, etc.), as well as provide other functionality such as:
measuring and graphing data collected from wireless earbuds to show progress
number of sleep hours
number of grinding/clenching events
duration of grinding/clenching events
pressure ranges of grinding/clenching events
volume adjustment
audible tone selections for earbuds
sleep sound selection with timer

The wireless ear buds may include a rechargeable batter that will charge inductively in a charging case.

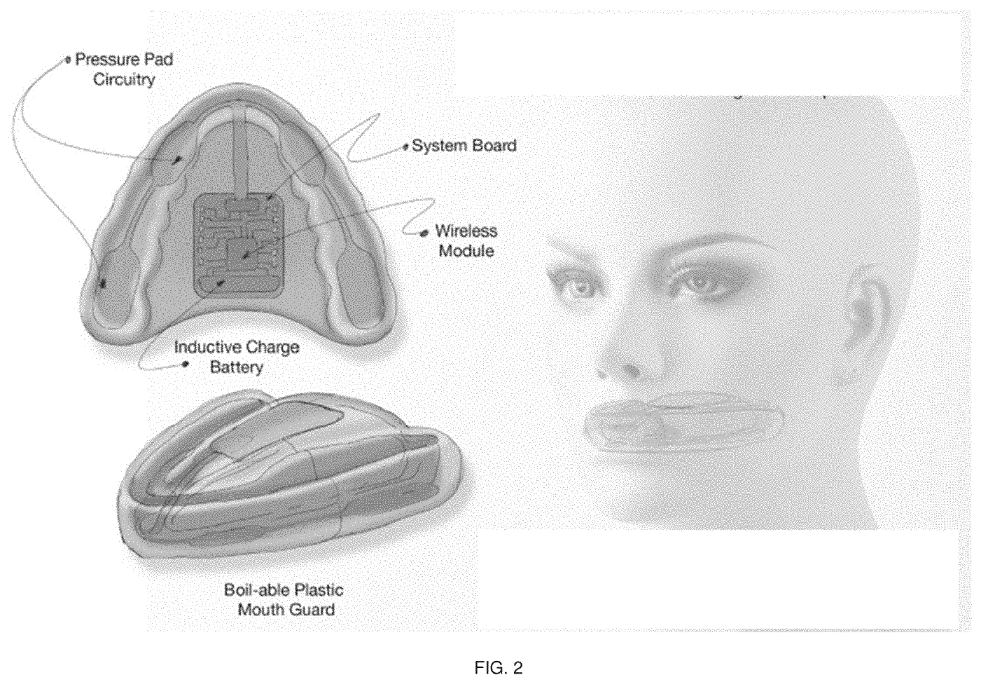

With reference to FIG. 2, an embodiment of a bruxism monitoring and prevention system provided in a mouth guard is illustrated, and may operate by detecting patient jaw clenching/teeth grinding via pressure on the mouthguard, while vibrating in order to attempt to wake the patient and/or otherwise stop the jaw clenching/teeth grinding.

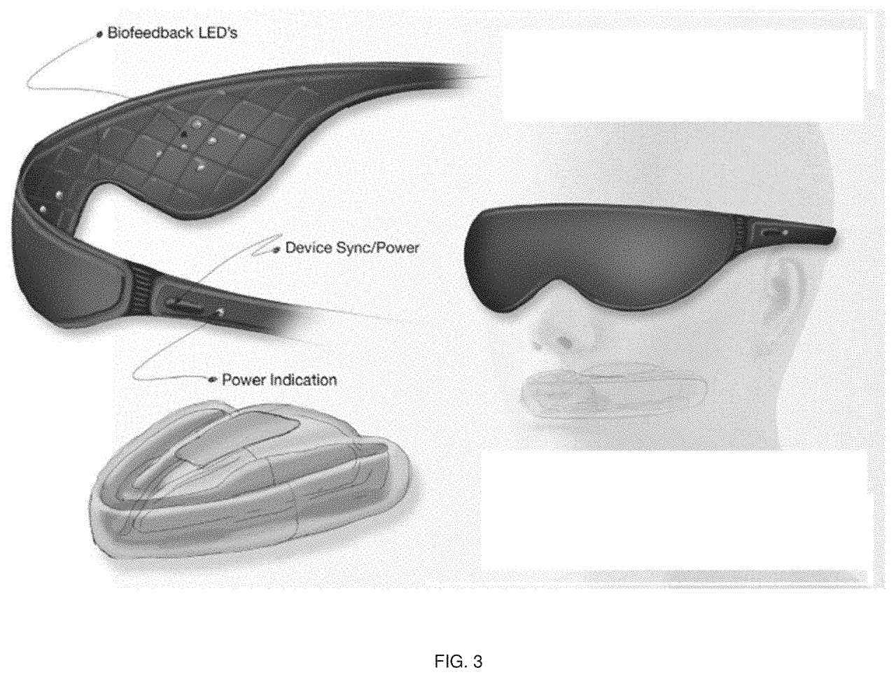

With reference to FIG. 3, an embodiment of a bruxism monitoring and prevention system is illustrated that utilizes a mouth guard that may be similar to the mouth guard illustrated in FIG. 2, along with an optical feedback eye mask. The bruxism monitoring and prevention may operate by detecting patient jaw clenching/teeth grinding via pressure on the mouthguard, while activating lights in the eye mask in order to attempt to wake the patient and/or otherwise stop the jaw clenching/teeth grinding.

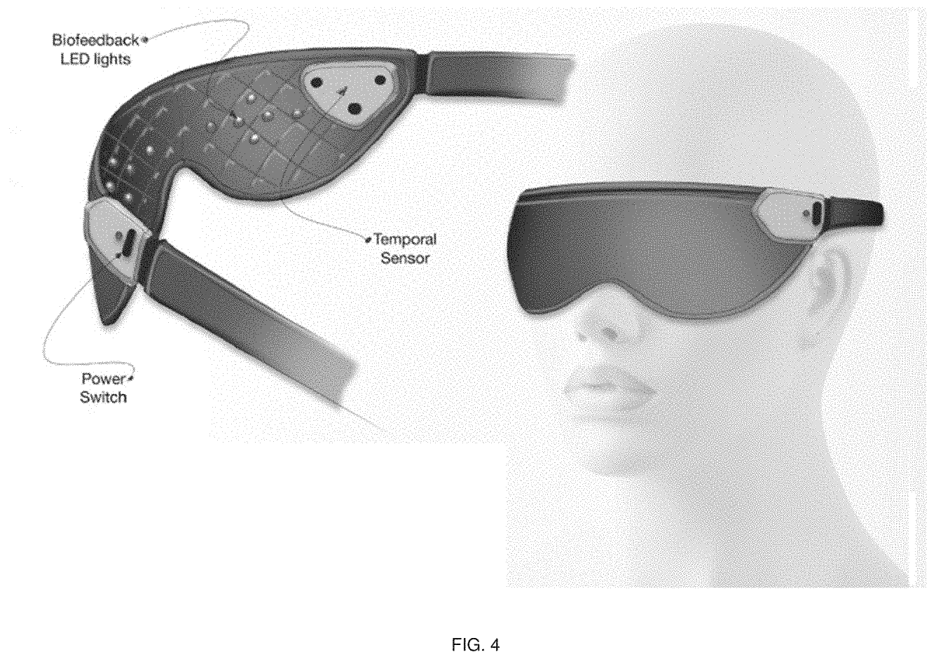

With reference to FIG. 4, an embodiment of a bruxism monitoring and prevention system is illustrated that is provided in an optical feedback eye mask, and may operate to detect patient jaw clenching/teeth grinding via temporal sensors on the eye mask positioned adjacent a patient's temples, while activating lights in the eye mask in order to attempt to wake the patient and/or otherwise stop the jaw clenching/teeth grinding.

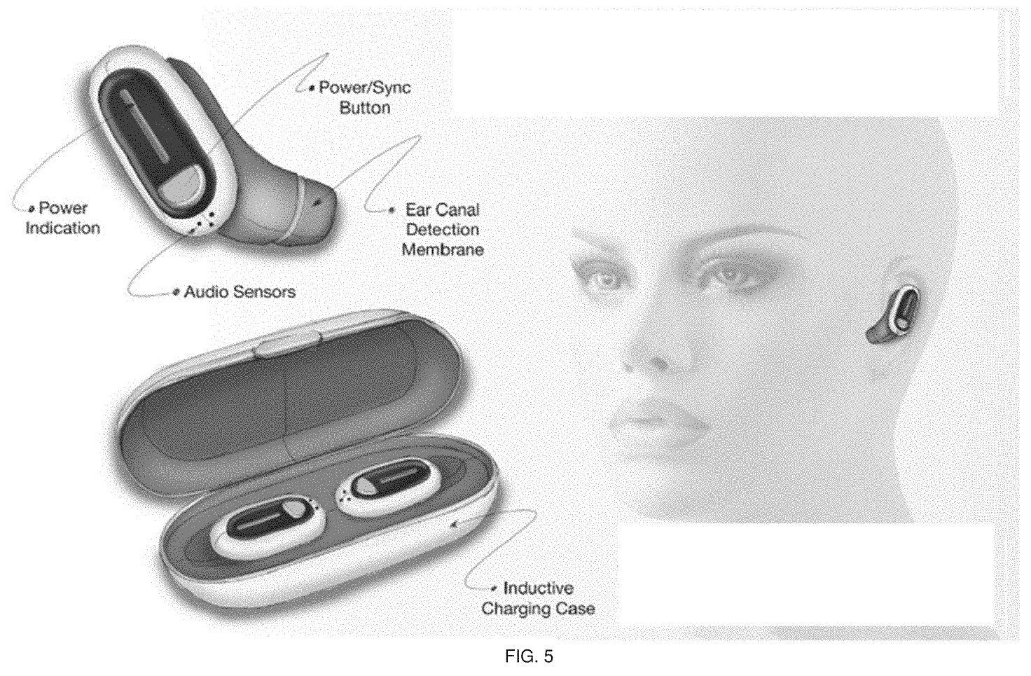

With reference to FIG. 5, an embodiment of a bruxism monitoring and prevention system is illustrated that is provided in an ear bud, and may operate to detect patient jaw clenching/teeth grinding via mechanisms that detections of changes in the size of the ear canal of the patient, while producing an audible tone or sound to attempt to wake the patient and/or otherwise stop the jaw clenching/teeth grinding.

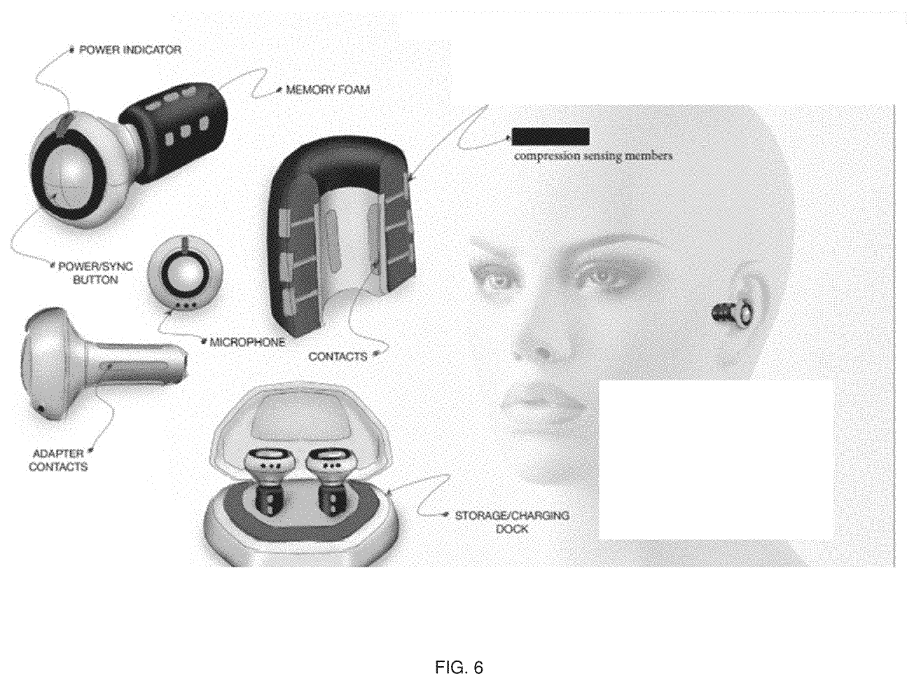

With reference to FIG. 6, an embodiment of a bruxism monitoring and prevention system is illustrated that is provided in an ear bud, and may operate to detect patient jaw clenching/teeth grinding via mechanisms that detections of changes in the size of the ear canal of the patient, while producing an audible tone or sound to attempt to wake the patient and/or otherwise stop the jaw clenching/teeth grinding.

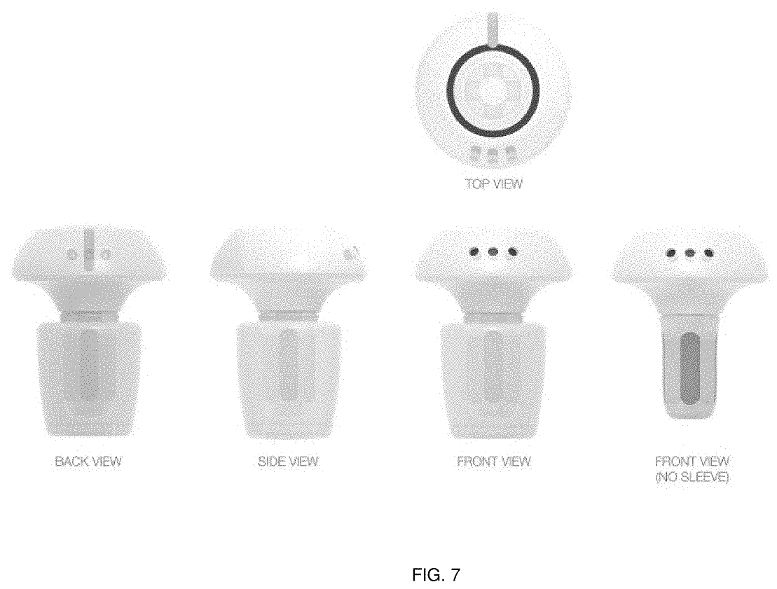

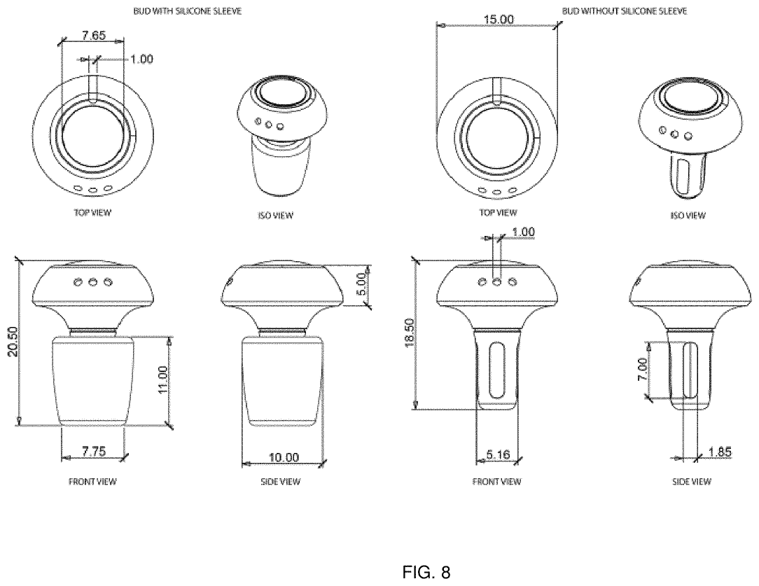

With reference to FIGS. 7 and 8, an embodiment of a bruxism monitoring and prevention system is illustrated that is provided in an ear bud, and may operate to detect patient jaw clenching/teeth grinding via mechanisms that detections of changes in the size of the ear canal of the patient, while producing an audible tone or sound to attempt to wake the patient and/or otherwise stop the jaw clenching/teeth grinding.

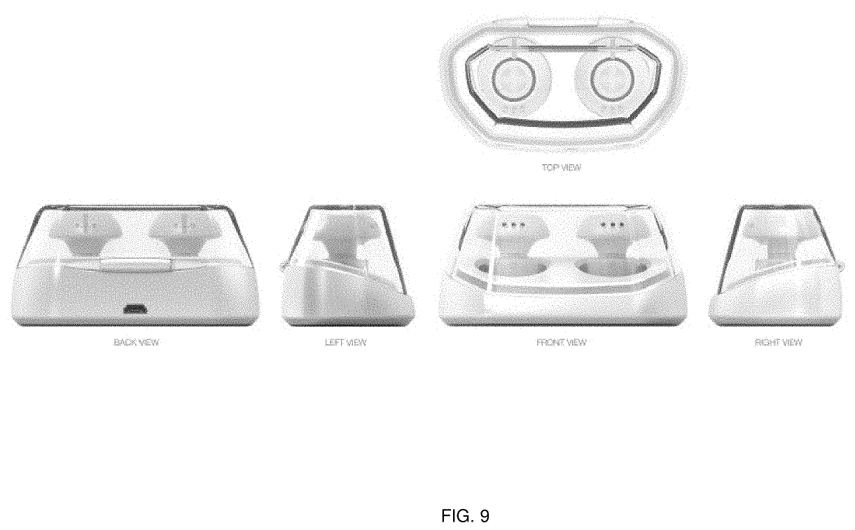

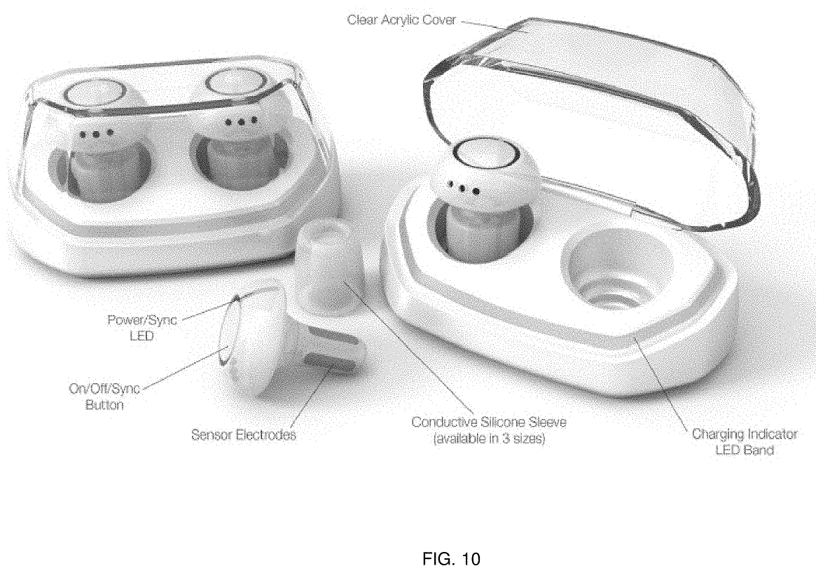

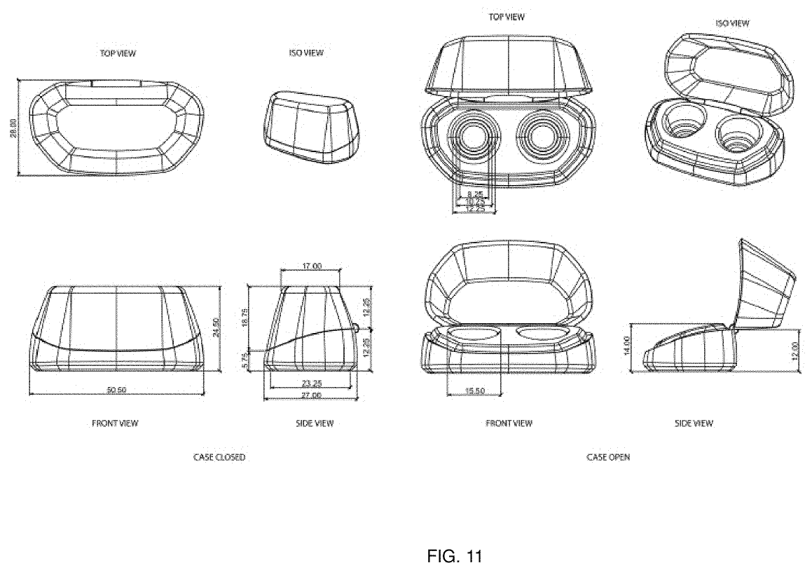

With reference to FIGS. 9, 10, and 11, an embodiment of a case is illustrated for the bruxism monitoring and prevention system provided in the ear bud illustrated in FIGS. 7 and 8.

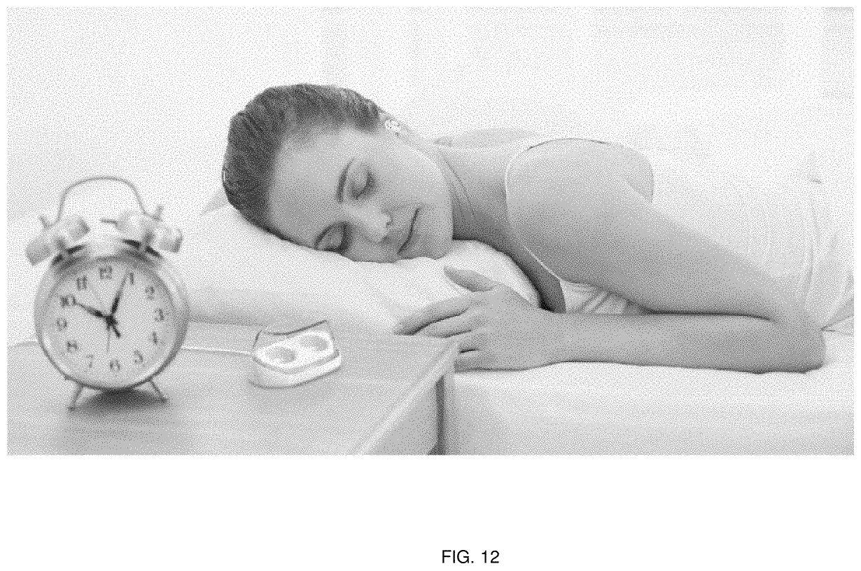

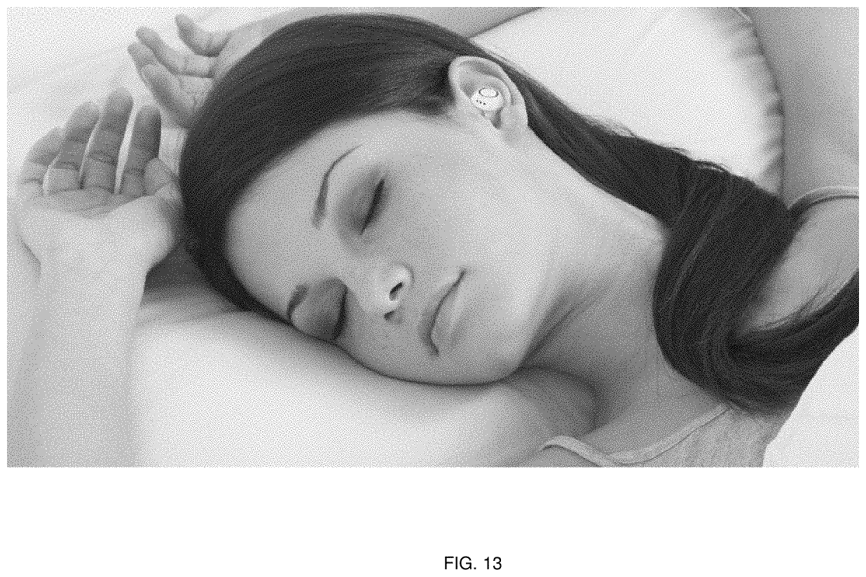

With reference to FIGS. 12 and 13, embodiments of patients are illustrated using the bruxism monitoring and prevention system provided in the ear bud illustrated in FIGS. 7 and 8.

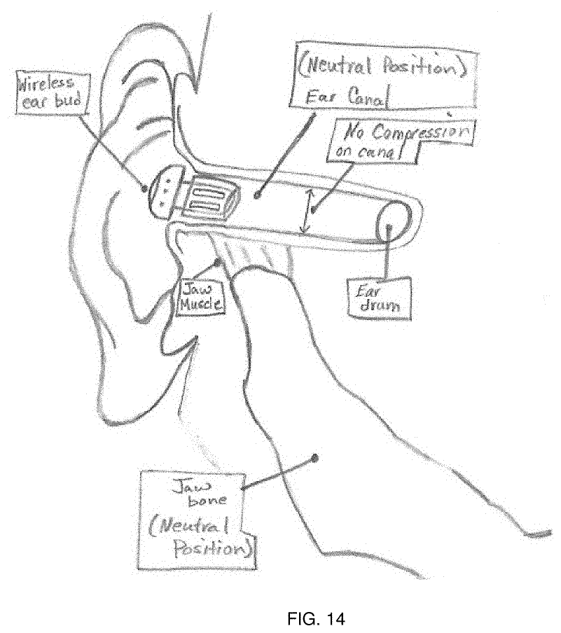

With reference to FIG. 14, an embodiment of a patient is illustrated using the bruxism monitoring and prevention system provided in the ear bud illustrated in FIGS. 7 and 8, and prior to a bruxism event.

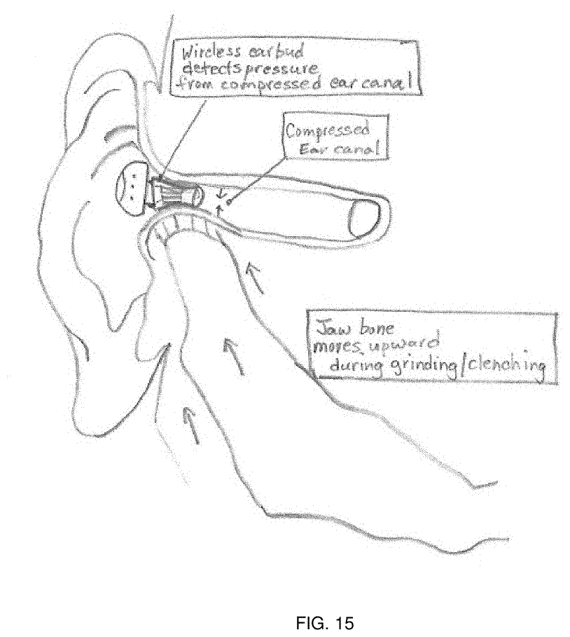

With reference to FIG. 15, an embodiment of a patient is illustrated using the bruxism monitoring and prevention system provided in the ear bud illustrated in FIGS. 7 and 8, and during a bruxism event.

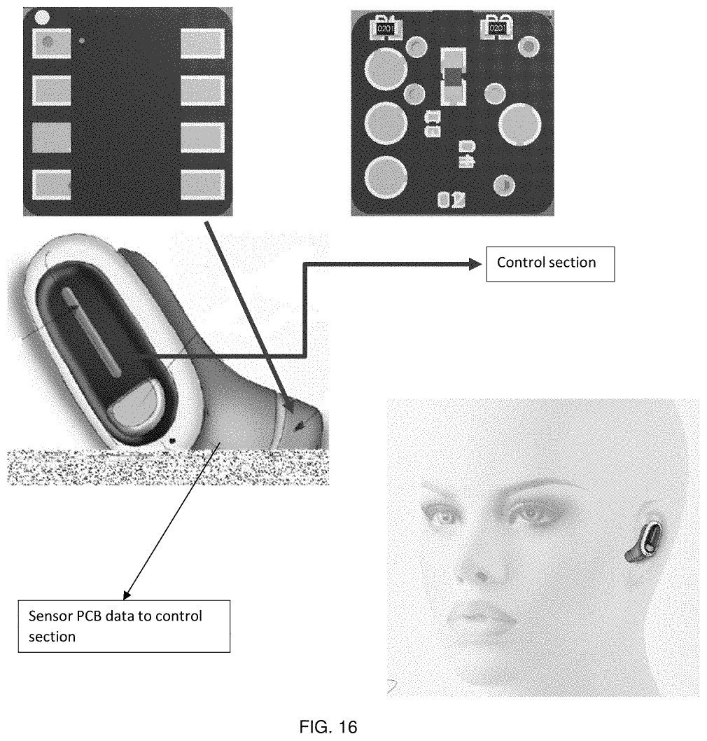

With reference to FIG. 16, an experimental embodiment of the bruxism monitoring and prevention system of the present disclosure is illustrated that shows an experimental embodiment of a Printed Circuit Board (PCB) that was fabricated with dimensions 5.5 mm×5.5 mm, and included a pressure sensor on one surface. The PCB may be placed inside the portion of the earbud that sits in a patients ear canal (illustrated), and used to detect the ear muscle movement associated with jaw clenching. As illustrated, a portion of the earbud may include control features (buttons, dials, etc.) to provide the patient control of the earbud.

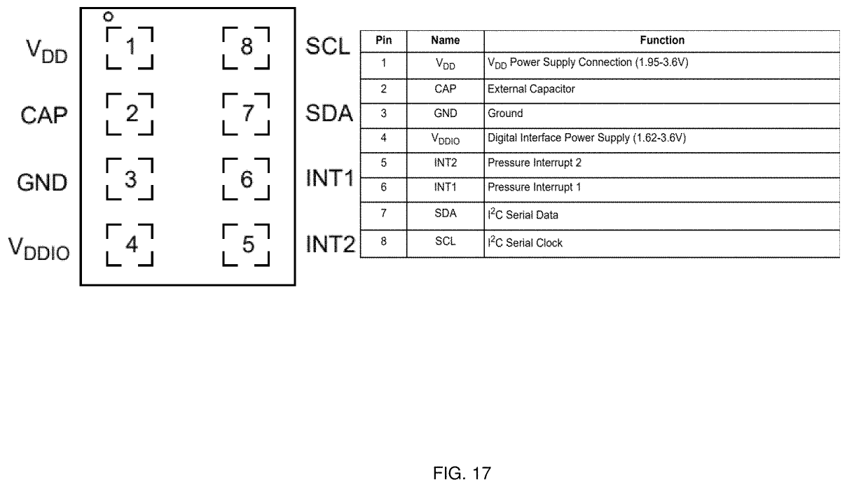

With reference to FIG. 17, an experimental embodiment of the bruxism monitoring and prevention system of the present disclosure is illustrated that shows a pin configuration utilized by an experimental embodiment of a pressure sensor provided by an MPL3115A2R1 pressure sensor available from NXP Semiconductors of Eindhoven, Netherlands. The MPL3115A2R1 pressure sensor is a low cost, low power and highly accurate pressure that is very sensitive and capable of detecting very small changes in pressure. The sensor outputs are digitized by a high resolution 24-bit ADC, and it has multiple user-programmable, power saving, interrupt and autonomous data acquisition modes. Features in the MPL3115A2R1 pressure sensor include:
1.95V to 3.6V Supply Voltage, internally regulated by LDO
1.6V to 3.6V Digital Interface Supply Voltage
Fully Compensated internally
Direct Reading, Compensated—Pressure: 20-bit measurement (Pascals)—Altitude: 20-bit measurement (meters)—Temperature: 12-bit measurement (degrees Celsius) Programmable Events
Autonomous Data Acquisition
Resolution down to 1 ft./30 cm
32 Sample FIFO
Ability to log data up to 12 days using the FIFO
1 second to 9-hour data acquisition rate
I2C digital output interface (operates up to 400 kHz)

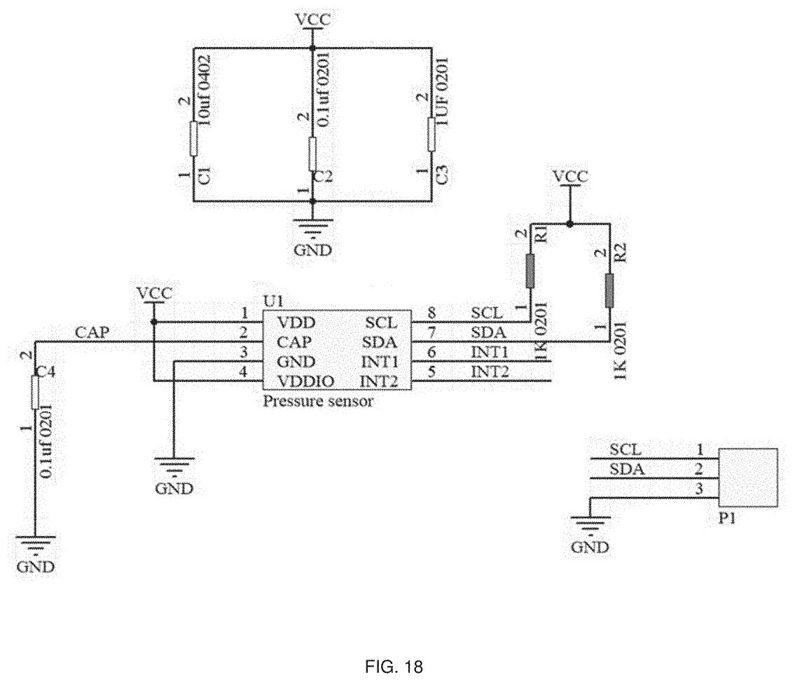

With reference to FIG. 18, an experimental embodiment of the bruxism monitoring and prevention system of the present disclosure is illustrated that shows an experimental embodiment of a sensor circuit diagram utilized for the system.

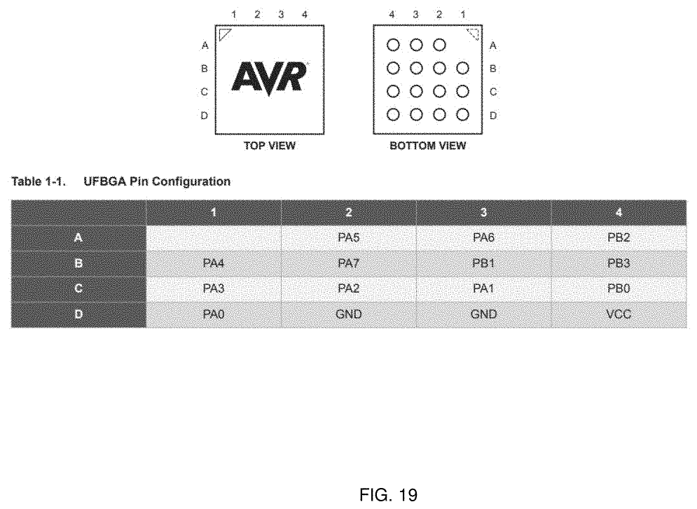

With reference to FIG. 19, an experimental embodiment of the bruxism monitoring and prevention system of the present disclosure is illustrated that shows an experimental embodiment of a processing unit provided by an ATTINY20 controller available from Microchip Technology of Chandler, Ariz., United States. The ATTINY20 controller is a small size, high performance and low power 8-bit AVR microcontroller, and FIG. 19 illustrates a UFBGA Pin configuration.

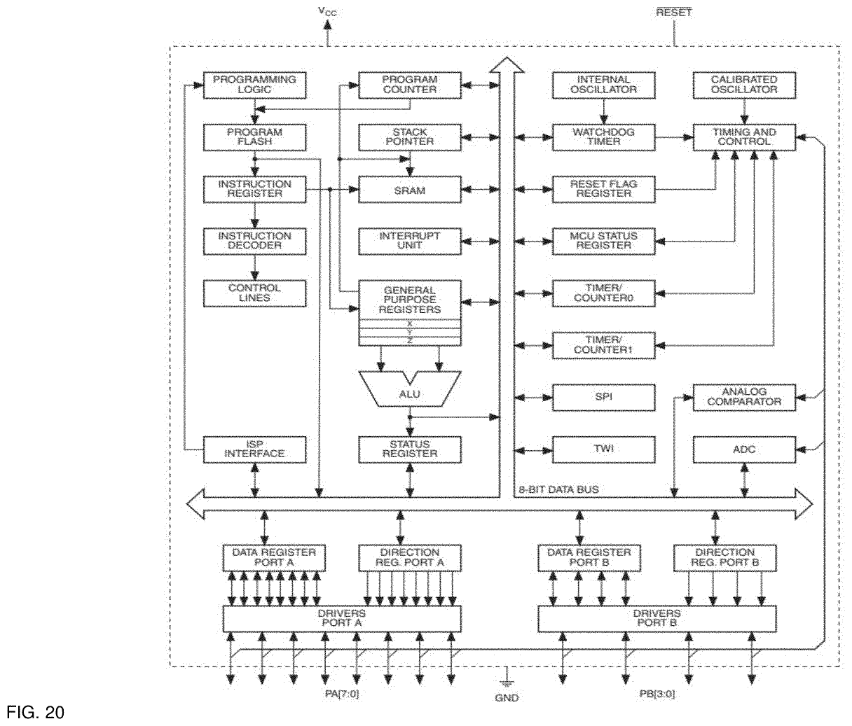

With reference to FIG. 20, an experimental embodiment of the bruxism monitoring and prevention system of the present disclosure is illustrated that shows a functional block diagram for the ATTINY20 microchip discussed above. A small size LIPO battery can be used inside the control section. As per the space available and battery capacity requirement, a LIPO battery can be customized to fit the earbud. A buzzer will be used by the ATTINY20 controller to inform user about the Bruxism.

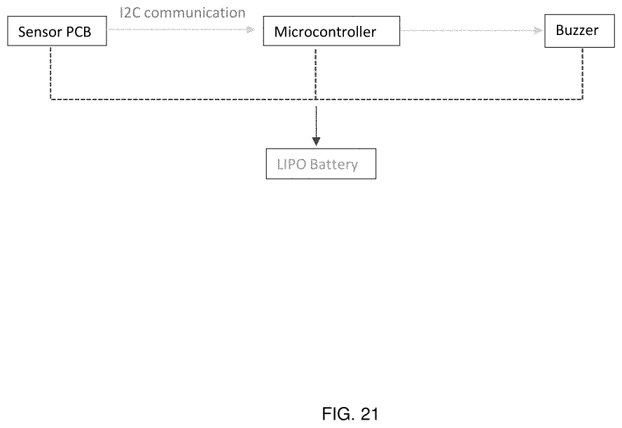

With reference to FIG. 21, an experimental embodiment of the bruxism monitoring and prevention system of the present disclosure is illustrated that shows how data from the pressure sensor PCB will be transmitted on the SDA/SCL pins of ATTINY20 microcontroller through I2C communication. The diameter of the ear canal decreases in response to how hard the jaw is clenched, and the sensor PCB will sense the contraction and relay the change in Pressure values to the ATTINY20 Controller. The ATTINY20 controller will use its Pre-build logics to determine the change in the values and produce an audible tone to gently remind the user to unclench and relax.

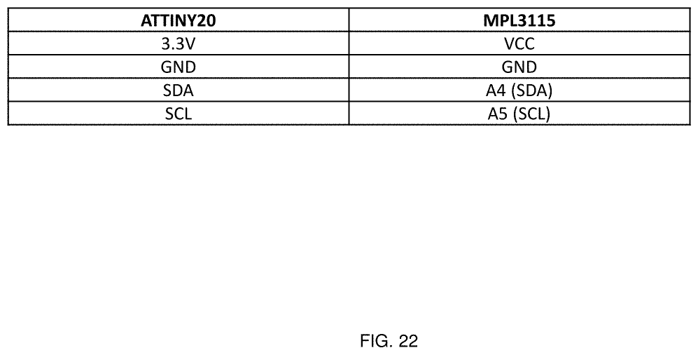

With reference to FIG. 22, an experimental embodiment of the bruxism monitoring and prevention system of the present disclosure is illustrated that shows the pin connections between the ATTINY20 microcontroller and the MPL3115 pressure sensor.

Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A bruxism monitoring and prevention system, comprising:
a bruxism detection subsystem that is dimensioned to be positioned at least partly in an ear canal of a patient, wherein the bruxism detection subsystem is configured:
detect, when the bruxism detection subsystem is positioned at least partly in the ear canal, a change in a size of the ear canal of the patient; and
generate, in response to detecting the change in the size of the ear canal, a bruxism event detection signal; and
a bruxism notification subsystem that is coupled to the bruxism detection subsystem, wherein the bruxism notification subsystem is configured to:
receive the bruxism event detection signal generated by the bruxism detection subsystem; and
generate, in response to receiving the bruxism event detection signal, a tone that is configured to be audible to the patient.
